(19) United States
(12) Patent Application Publication
(10) Pub. No.: US 2025/0204553 A1
(43) Pub. Date: Jun. 26, 2025

(54) FEED AND METHODS FOR CONTROLLING HEARTWORM INFECTIONS IN A MAMMAL

(71) Applicant: In the Bowl Animal Health, Inc., Nashiville, TN (US)

(72) Inventors: Robin S. Readnour, Nashville, TN (US); Joseph R. Winkle, Carmel, IN (US); Daniel E. Snyder, Indianapolis, IN (US); Kevin E. Willard, Knightstown, IN (US)

(21) Appl. No.: 19/077,569

(22) Filed: Mar. 12, 2025

Related U.S. Application Data
(63) Continuation of application No. PCT/US2023/074371, filed on Sep. 15, 2023.
(60) Provisional application No. 63/407,140, filed on Sep. 15, 2022.

(51) Int. Cl. A23K 20/121; A23K 20/111; A23K 50/40

(52) U.S. Cl. CPC A23K 20/121; A23K 20/111; A23K 50/40

(57) ABSTRACT

An oral animal feed and a method of preventing and/or controlling heartworms in a mammal in need thereof by orally administering to the mammal, for example a canine, a feed comprising an effective amount of a systemic anthelmintic that has a relatively long half-life for an effective time to thereby cause the amount of the anthelmintic in the mammal's blood to rise to and be maintained at a therapeutically effective level for preventing and/or controlling heartworms.

### RELATED APPLICATIONS

This application claims priority to U.S. Provisional Patent Application No. 63/407,140, filed Sep. 15, 2022, and PCT Application No. PCT/US2023/74371, filed Sep. 15, 2023, both of which are hereby incorporated herein by reference.

### SUMMARY

The teachings of this disclosure generally relate to a method of administering an active material to control heartworms in mammals.

There are five main types of parasitic worms, or helminths, that most commonly infect domestic dogs: ascarid worms, tapeworms, hookworms, whipworms and heartworms. These helminths can cause serious health problems, including gastro-intestinal issues, abdominal pain, weight loss, dehydration, anemia, pneumonia and death in severe cases. Further, some helminths can be readily transmitted by insect bites. For example, Dirofilaria immitis, more commonly known as heartworm, which infects dogs, cats, ferrets, and some other animals, can be readily transmitted by insect bites and is often fatal if left untreated. In some instances, the treatment for an adult heartworm infection may itself be fatal for the animal. Thus, it is essential that dogs, cats, and ferrets kept as pets receive appropriate veterinary care, including routine treatments to prevent infection with heartworms.

Treatments currently available for controlling helminth infections in animals are known to achieve varying degrees of success. Issues that contribute to ineffective control of heartworms in, for example, dogs and cats include improper oral dosing that may further lead to anthelmintic resistance and vomiting caused by the high doses required for some anthelmintics to achieve effectiveness.

Embodiment 1: A method of controlling heartworm infections, comprising:
administering a cumulative therapeutically effective amount of a least one systemic anthelmintic to a mammal in need thereof, the cumulative therapeutically effective amount comprising: a plurality of individual micro-doses of the at least one systemic anthelmintic,
wherein the micro-doses are administered to the mammal daily or substantially daily, wherein the at least one anthelmintic has a half-life in the mammal of at least at least 4 days, or at least 5 days, or at least 6, days or at least 7, or at least 10 days.

Embodiment 2: The method according to embodiment 1, wherein the systemic anthelmintic is an integral part of at least one of a medicated feed, a chew, a snack, a tablet, a capsule, a powder, a slave, a suspension, a solution, and a paste.

Embodiment 3: The method according to embodiments 1-2, wherein the systemic anthelmintic is an integral part of a medicated pet feed and said medicated feed is a consumable selected from the group consisting of a kibble, a wet food, a paste, a chew, and a treat.

Embodiment 4: The method according to embodiments 1-3, wherein in the mammal is selected from the group consisting of canines and felines.

Embodiment 5: The method according to embodiment 4, wherein the mammal is a domesticated dog.

Embodiment 6: The method according to embodiment 4, wherein the mammal is a domesticated cat.

Embodiment 7: The method according to embodiments 1-6, wherein the cumulative therapeutically effective amount is sufficient to prevent an infection of at least one helminth selected from the group consisting of: Dirofilaria immitis, Aelurostrongylus abstrusus, Angiostrongylus vasorum, Crenosoma vulpis, and Eucoleus aerophilu.

Embodiment 8: The method according to embodiments 1-6, wherein the cumulative therapeutically effective amount is sufficient to control an infection of at least one helminth selected from the group consisting of: Dirofilaria immitis, Aelurostrongylus abstrusus, Angiostrongylus vasorum, Crenosoma vulpis, and Eucoleus aerophilu.

Embodiment 9: The method according to embodiment 1-8, wherein the systemic anthelmintic is a milbemycin.

Embodiment 10: The method according to embodiment 9, wherein the milbemycin is moxidectin or a therapeutically acceptable salt thereof.

Embodiment 11: The method according to embodiments 1-10, wherein the cumulative therapeutically effective amount of moxidectin administered to the mammal over a course of about a 30 day period does not exceed 48 μg of moxidectin per kg of mammal body weight.

Embodiment 12: The method according to embodiment 11, wherein the amount of moxidectin in the micro-dose is in a range selected from the group consisting of: about 0.2 μg/kg to about 1.6 μg/kg; about 0.4 μg/kg to about 1.2 μg/kg; about 0.6 μg/kg to about 1.0 μg/kg, about 0.8 μg/kg to about 0.9 μg/kg.

Embodiment 13: The method according to embodiments 11-12, wherein the amount of moxidectin in the micro-dose is selected from the group consisting of about 0.2 μg/kg, about 0.4 μg/kg, about 0.6 μg/kg, about 0.8 μg/kg and about 1.6 μg/kg.

Embodiment 14: The method of any of embodiments 10-13, wherein said administration provides a concentration of moxidectin of more than about 7.8 ng/ml and less than about 190 ng/ml in said mammal's blood for at least 30 days.

Embodiment 15: The method of any of embodiments 10-13, wherein said administration provides a concentration of moxidectin of more than about 7.8 ng/ml and less than about 100 ng/ml in said mammal's blood for at least 30 days.

Embodiment 16: The method according to embodiment 1-8, wherein the systemic

anthelmintic is an amino-acetonitrile derivative or a therapeutically acceptable salt thereof.

Embodiment 17: The method according to embodiment 16, wherein the amino-acetonitrile derivative is monepantel or a therapeutically acceptable salt thereof.

Embodiment 18: The method according to embodiment 17, wherein the cumulative therapeutically effective amount of monepantel administered to the mammal over a course of about a 30 day period does not exceed 25 mg of monepantel per kg of mammal body weight.

Embodiment 19: The method according to embodiments 17-18, wherein the amount of monepantel in the micro-dose is in a range selected from the group consisting of: about 0.025 mg/kg to about 0.83 mg/kg; about 0.05 mg/kg to about 0.5 mg/kg; about 0.125 mg/kg to about 0.25 mg/kg.

Embodiment 20: The method according to embodiment 17-19, wherein the amount of montepantel in the micro-dose is selected from the group consisting of: about 0.025 mg/kg; about 0.05 mg/kg; about 0.125 mg/kg; about 0.25 mg/kg; about 0.5 mg/kg; and about 0.83 mg/kg.

Embodiment 21: The method according to embodiments 1-20, wherein the number of days between administering any two micro-doses is less than a period of 2 days, 3 days, 4 days, 5 days, 6 days or 7 days.

Embodiment 22: The method according to embodiments 1-21, wherein the at least one micro-dose is administered orally.

Embodiment 23: The method according to embodiments 1-22, further comprising administering at least one additional anthelmintic.

Embodiment 24: The method according to embodiment 23, wherein the at least one additional anthelmintic is a systemic anthelmintic.

Embodiment 25: The method according to embodiments 23-24, wherein the at least one additional anthelmintic has a half-life of less than 1 day.

Embodiment 26: The method according to embodiment 23, wherein the at least one additional anthelmintic is selected from the group consisting of: at least one compound that blocks acetylcholine receptors; at least one compound that acts on the nicotinic acetylcholine receptor ACR-23, and at least one compound that acts on the calcium-gated potassium channel SLO-1.

Embodiment 27: The method according to embodiment 23, wherein the at least one additional anthelmintic is at least one compound selected from the group consisting of: a macrocylic lactone or a salt or a derivative thereof; a cyclooctadepsipeptide or a salt or a derivative thereof; a tetrahydropyrimidine or a salt or a derivative thereof; an imidazothiazole or a salt or a derivative thereof; a paraherquamide or a salt or a derivative thereof; an aminophenylamidine or a salt or a derivative thereof; an organophosphates or a salt or a derivative thereof; a substituted phenyl or a salt or a derivative thereof; a piperazinone or a salt or a derivative thereof; and an octadepsipeptide or a salt or a derivative thereof.

Embodiment 28: The method according to embodiment 27, wherein the at least one additional anthelmintic includes macrocyclic lactone and the macrocyclic lactone is at least one compound selected from the group consisting of: abamectin, doramectin, emamectin, eprinomectin, ivermectin, milbemycin oxime, moxidectin, nemadectin, selamectin; the benzimidazoles and probenzimidazoles, such as albendazole, albendazole-sulphoxide, cambendazole, cyclobendazole, febantel, fenbendazole, flubendazole, mebendazole, netobimin, oxfendazole, oxibendazole, parbendazole, thiabendazole, thiophanate, and triclabendazole and a salt or a derivative of any of the foregoing.

Embodiment 29: The method according to embodiment 27, wherein the at least one additional anthelmintic includes cyclooctadepsipeptide and the cyclooctadepsipeptide is at least one compound selected from the group consisting of: emodepside, PF1022 and a salt or a derivative of any of the foregoing.

Embodiment 30: The method according to embodiment 27, wherein the at least one additional anthelmintic includes amino-acetonitrile derivative and the amino-acetonitrile derivative is monepantel or a salt or a derivative thereof.

Embodiment 31: The method according to embodiment 27, wherein the at least one additional anthelmintic includes tetrahydropyrimidine and the tetrahydropyrimidine is at least one compound selected from the group consisting of: morantel, pyrantel, and oxantel and a salt or a derivative of any of the foregoing.

Embodiment 32: The method according to embodiment 27, wherein the at least one additional anthelmintic includes imidazothiazole and the imidazothiazole is at least one compound selected from the group consisting of: butamisole, levamisole, tetramisole and a salt or a derivative of any of the foregoing.

Embodiment 33: The method according to embodiment 27, wherein the at least one additional anthelmintic includes paraherquamide and the paraherquamide is at least one compound selected from the group consisting of: derquantel, paraherquamide and a salt or a derivative of any of the foregoing.

Embodiment 34: The method according to embodiment 27, wherein the at least one additional anthelmintic includes aminophenylamidine and the aminophenylamidine is at least one compound selected from the group consisting of: amidantel, deacylated amidantel (dAMD), tribendimidine and a salt or a derivative of any of the foregoing.

Embodiment 35: The method according to embodiment 27, wherein the at least one additional anthelmintic includes organophosphate and the organophosphate is at least one compound selected from the group consisting of: coumaphos, crufomate, dichlorvos, haloxone, naphthalofos, trichlorfon and a salt or a derivative of any of the foregoing.

Embodiment 36: wherein the at least one additional anthelmintic includes substituted phenyl and the substituted phenyl is at least one compound selected from the group consisting of: bithionol, disophenol, hexachlorophene, niclofolan, meniclopholan, nitroxynil and a salt or a derivative of any of the foregoing.

Embodiment 37: The method according to embodiment 27, wherein the at least one additional anthelmintic includes piperazinone and the piperazinone is at least one compound selected from the group consisting of: praziquantel, epsiprantel and a salt or a derivative of any of the foregoing.

Embodiment 38: The method according to embodiment 23, wherein the at least one additional anthelmintic is a compound selected from the group consisting of: amoscanate, bephenium, bunamidine, clonazepam, clorsulon, diamfenetid, dichlorophen, diethylcarbamazine, emetine, hetolin, hycanthone, lucanthone, Miracil, mirasan, niclosamide, niridazole, nitroxynil, nitroscanate, oltipraz, omphalotin, oxamniquin, paromomycin, piperazine, resorantel, salicylanilides, bromoxanide, brotianide, clioxanide, closantel, niclosamide, oxyclozanide, rafoxanide, tribromsalan and a salt or a derivative of any of the foregoing.

Embodiment 39: The method according to embodiments 23, wherein the at least one additional anthelmintic is diethylcarbamazine or a salt or a derivative thereof.

Embodiment 40: A composition for controlling heartworm infections, comprising:
a micro-dose of at least one systemic anthelmintic, said at least one systemic anthelmintic being active in a mammal and having a half-life in the system of the mammal of at least 4 days, or at least 5 days, or at least 6, days or at least 7, or at least 10 days, and a cumulative dose of a plurality of said micro-doses is sufficient to control a heartworm infection.

Embodiment 41: The composition according to embodiment 40, wherein the systemic anthelmintic is an integral part of at least one formulation selected from the group consisting of: a medicated feed, a treat, a chew, a snack, a tablet, a capsule, a powder, a salve, a suspension, a solution, a sachet, and a paste, or an integral part thereof.

Embodiment 42: The composition of embodiment 40, wherein the medicated feed includes at least one feedstuff, wherein the micro-dose of the systemic anthelmintic is an integral part of the feed stuff.

Embodiment 43: The composition according to embodiment 40, wherein the medicated feed is selected from the group of formulations consisting of a kibble, a chew, a wet food, a paste and a snack.

Embodiment 44: The composition according to embodiments 40-43, wherein the mammal is selected from the group consisting of canines and felines.

Embodiment 45: The composition according to embodiment 44, wherein the mammal is a domesticated dog.

Embodiment 46: The composition according to embodiment 44, wherein the mammal is a domesticated cat.

Embodiment 47: The composition according to embodiments 40-46, wherein the cumulative therapeutically effective amount is sufficient to prevent an infection of at least one helminth selected from the group consisting of: Dirofilaria immitis, Aelurostrongylus abstrusus, Angiostrongylus vasorum, Crenosoma vulpis, and Eucoleus aerophilu.

Embodiment 48: The composition according to embodiments 40-46, wherein the cumulative therapeutically effective amount is sufficient to control an infection of at least one helminth selected from the group consisting of: Dirofilaria immitis, Aelurostrongylus abstrusus, Angiostrongylus vasorum, Crenosoma vulpis, and Eucoleus aerophilu.

Embodiment 49: The composition according to embodiments 44-48, wherein the systemic anthelmintic is a milbemycin.

Embodiment 50: The composition according to embodiment 49, wherein the milbemycin is moxidectin or a therapeutically acceptable salt thereof.

Embodiment 51: The composition according to embodiment 50, wherein the amount of moxidectin in the micro-dose is in a range selected from the group consisting of: about 0.2 μg/kg to about 1.6 μg/kg; about 0.4 μg/kg to about 1.2 μg/kg; about 0.6 μg/kg to about 1.0 μg/kg, about 0.8 μg/kg to about 0.9 μg/kg.

Embodiment 52: The composition according to embodiments 50-51, wherein the amount of moxidectin in the micro-dose is selected from the group consisting of about 0.2 μg/kg, about 0.4 μg/kg, about 0.6 μg/kg, about 0.8 μg/kg and about 1.6 μg/kg.

Embodiment 53: The composition of any of embodiments 50-52, wherein said administration provides a concentration of moxidectin of more than about 7.8 ng/mL and less than about 190 ng/ml in said mammal's blood for at least 30 days.

Embodiment 54: The composition of any of embodiments 50-53, wherein said administration provides a concentration of moxidectin of more than about 7.8 ng/ml and less than about 100 ng/ml in said mammal's blood for at least 30 days.

Embodiment 55: The composition according to embodiments 40-48, wherein the systemic anthelmintic is an amino-acetonitrile derivative or a therapeutically acceptable salt

Embodiment 56: The composition according to embodiment 55, wherein the amino-acetonitrile derivative is monepantel or a therapeutically acceptable salt thereof.

Embodiment 57: The composition according to embodiment 56, wherein the amount of monepantel in the micro-dose is in a range selected from the group consisting of: about 0.025 mg/kg to about 0.83 mg/kg; about 0.05 mg/kg to about 0.5 mg/kg; about 0.125 mg/kg to about 0.25 mg/kg.

Embodiment 58: The composition according to embodiments 56-57, wherein the amount of montepantel in the micro-dose is selected from the group consisting of: about 0.025 mg/kg; about 0.05 mg/kg; about 0.125 mg/kg; about 0.25 mg/kg; about 0.5 mg/kg; and about 0.83 mg/kg.

Embodiment 59: The composition according to embodiments 40-58, wherein the at least one micro-dose is formulated to be administered orally.

Embodiment 60: The composition according to embodiments 40-59, further comprising at least one additional anthelmintic.

Embodiment 61: The composition according to embodiment 60, wherein the at least one additional anthelmintic is a systemic anthelmintic.

Embodiment 62: The composition according to embodiments 60-61, wherein the at least one additional anthelmintic has a half-life of less than 1 day.

Embodiment 63: The composition according to embodiment 60, wherein the at least one additional anthelmintic is selected from the group consisting of: at least one compound that blocks acetylcholine receptors; at least one compound that acts on the nicotinic acetylcholine receptor ACR-23, and at least one compound that acts on the calcium-gated potassium channel SLO-1.

Embodiment 64: The composition according to embodiment 60, wherein the at least one additional anthelmintic is at least one compound selected from the group consisting of: a macrocylic lactone or a salt or a derivative thereof; a cyclooctadepsipeptide or a salt or a derivative thereof; a tetrahydropyrimidine or a salt or a derivative thereof; an imidazothiazole or a salt or a derivative thereof; a paraherquamide or a salt or a derivative thereof; an aminophenylamidine or a salt or a derivative thereof; an organophosphates or a salt or a derivative thereof; a substituted phenyl or a salt or a derivative thereof; a piperazinone or a salt or a derivative thereof; and an octadepsipeptide or a salt or a derivative thereof.

Embodiment 65: The composition according to embodiment 64, wherein the at least one additional anthelmintic includes macrocyclic lactone and the macrocyclic lactone is at least one compound selected from the group consisting of: abamectin, doramectin, emamectin, eprinomectin, ivermectin, milbemycin oxime, moxidectin, nemadectin, selamectin; the benzimidazoles and probenzimidazoles, such as albendazole, albendazole-sulphoxide, cambendazole, cyclobendazole, febantel, fenbendazole, flubendazole, mebendazole, netobimin, oxfendazole, oxibendazole, parbendazole, thiabendazole, thiophanate, and triclabendazole and a salt or a derivative of any of the foregoing.

Embodiment 66: The composition according to embodiment 64, wherein the at least one additional anthelmintic includes cyclooctadepsipeptide and the cyclooctadepsipeptide is at least one compound selected from the group consisting of: emodepside, PF1022 and a salt or a derivative of any of the foregoing.

Embodiment 67: The composition according to embodiment 64, wherein the at least one additional anthelmintic includes amino-acetonitrile derivative and the amino-acetonitrile derivative is monepantel or a salt or a derivative thereof.

Embodiment 68: The composition according to embodiment 64, wherein the at least one additional anthelmintic includes tetrahydropyrimidine and the tetrahydropyrimidine is at least one compound selected from the group consisting of: morantel, pyrantel, and oxantel and a salt or a derivative of any of the foregoing.

Embodiment 69: The composition according to embodiment 64, wherein the at least one additional anthelmintic includes imidazothiazole and the imidazothiazole is at least one compound selected from the group consisting of: butamisole, levamisole, tetramisole and a salt or a derivative of any of the foregoing.

Embodiment 70: The composition according to embodiment 64, wherein the at least one additional anthelmintic includes paraherquamide and the paraherquamide is at least one compound selected from the group consisting of: derquantel, paraherquamide and a salt or a derivative of any of the foregoing.

Embodiment 71: The composition according to embodiment 64, wherein the at least one additional anthelmintic includes aminophenylamidine and the aminophenylamidine is at least one compound selected from the group consisting of: amidantel, deacylated amidantel (dAMD), tribendimidine and a salt or a derivative of any of the foregoing.

Embodiment 72: The composition according to embodiment 64, wherein the at least one additional anthelmintic includes organophosphate and the organophosphate is at least one compound selected from the group consisting of: coumaphos, crufomate, dichlorvos, haloxone, naphthalofos, trichlorfon and a salt or a derivative of any of the foregoing.

Embodiment 73: The composition according to embodiment 64, wherein the at least one additional anthelmintic includes substituted phenyl and the substituted phenyl is at least one compound selected from the group consisting of: bithionol, disophenol, hexachlorophene, niclofolan, meniclopholan, nitroxynil and a salt or a derivative of any of the foregoing.

Embodiment 74: The composition according to embodiment 64, wherein the at least one additional anthelmintic includes piperazinone and the piperazinone is at least one compound selected from the group consisting of: praziquantel, epsiprantel and a salt or a derivative of any of the foregoing.

Embodiment 75: The composition according to embodiment 60, wherein the at least one additional anthelmintic is a compound selected from the group consisting of: amoscanate, bephenium, bunamidine, clonazepam, clorsulon, diamfenetid, dichlorophen, diethylcarbamazine, emetine, hetolin, hycanthone, lucanthone, Miracil, mirasan, niclosamide, niridazole, nitroxynil, nitroscanate, oltipraz, omphalotin, oxamniquin, paromomycin, piperazine, resorantel, salicylanilides, bromoxanide, brotianide, clioxanide, closantel, niclosamide, oxyclozanide, rafoxanide, tribromsalan and a salt or a derivative of any of the foregoing.

Embodiment 76: The composition according to embodiment 60, wherein the at least one additional anthelmintic is diethylcarbamazine or a salt or a derivative thereof.

Embodiment 77: A medicated feed for use in a method of controlling heartworm infections, comprising: administering a cumulative therapeutically effective amount of a least one systemic anthelmintic to a mammal in need thereof, the cumulative therapeutically effective amount comprising: a plurality of individual micro-doses of the at least one systemic anthelmintic,
wherein the micro-doses are administered to the mammal daily or substantially daily, wherein the at least one anthelmintic has a half-life in the mammal of at least at least 4 days, or at least 5 days, or at least 6, days or at least 7, or at least 10 days.

Embodiment 78: The medicated feed for use in the method according to embodiment 77, wherein the systemic anthelmintic is an integral part of at least one of a medicated feed, a chew, a snack, a tablet, a capsule, a powder, a slave, a suspension, a solution, and a paste.

Embodiment 79: The medicated feed for use in the method according to embodiments 77-78, wherein the systemic anthelmintic is an integral part of a medicated pet feed and said medicated feed is a consumable selected from the group consisting of a kibble, a wet food, a paste, a chew, and a treat.

Embodiment 80: The medicated feed for use in the method according to embodiments 77-79, wherein in the mammal is selected from the group consisting of canines and felines.

Embodiment 81: The medicated feed for use in the method according to embodiment 80, wherein the mammal is a domesticated dog.

Embodiment 82: The medicated feed for use in the method according to embodiment 80, wherein the mammal is a domesticated cat.

Embodiment 83: The medicated feed for use in the method according to embodiments 77-82, wherein the cumulative therapeutically effective amount is sufficient to prevent an infection of at least one helminth selected from the group consisting of: Dirofilaria immitis, Aelurostrongylus abstrusus, Angiostrongylus vasorum, Crenosoma vulpis, and Eucoleus aerophilu.

Embodiment 84: The medicated feed for use in the method according to embodiments 77-82, wherein the cumulative therapeutically effective amount is sufficient to control an infection of at least one helminth selected from the group consisting of: Dirofilaria immitis, Aelurostrongylus abstrusus, Angiostrongylus vasorum, Crenosoma vulpis, and Eucoleus aerophilu.

Embodiment 85: The medicated feed for use in the method according to embodiment 77-84, wherein the systemic anthelmintic is a milbemycin.

Embodiment 86: The medicated feed for use in the method according to embodiment 85, wherein the milbemycin is moxidectin or a therapeutically acceptable salt

Embodiment 87: The medicated feed for use in the method according to embodiments 85-86, wherein the cumulative therapeutically effective amount of moxidectin administered to the mammal over a course of about a 30 day period does not exceed 48 μg of moxidectin per kg of mammal body weight.

Embodiment 88: The medicated feed for use in the method according to embodiment 87, wherein the amount of moxidectin in the micro-dose is in a range selected from the group consisting of: about 0.2 μg/kg to about 1.6 μg/kg; about 0.4 μg/kg to about 1.2 μg/kg; about 0.6 μg/kg to about 1.0 μg/kg, about 0.8 μg/kg to about 0.9 μg/kg.

Embodiment 89: The medicated feed for use in the method according to embodiments 85-88, wherein the amount of moxidectin in the micro-dose is selected from the group consisting of about 0.2 μg/kg, about 0.4 μg/kg, about 0.6 μg/kg, about 0.8 μg/kg and about 1.6 μg/kg.

Embodiment 90: The medicated feed for use in the method of any of embodiments 85-89, wherein said administration provides a concentration of moxidectin of more than about 7.8 ng/ml and less than about 190 ng/ml in said mammal's blood for at least 30 days.

Embodiment 91: The medicated feed for use in the method of any of embodiments 85-89, wherein said administration provides a concentration of moxidectin of more than about 7.8 ng/ml and less than about 100 ng/ml in said mammal's blood for at least 30 days.

Embodiment 92: The medicated feed for use in the method according to embodiment 77-84, wherein the systemic anthelmintic is an amino-acetonitrile derivative or a therapeutically acceptable salt thereof.

Embodiment 93: The medicated feed for use in the method according to embodiment 92, wherein the amino-acetonitrile derivative is monepantel or a therapeutically acceptable salt thereof.

Embodiment 94: The medicated feed for use in the method according to embodiment 93, wherein the cumulative therapeutically effective amount of monepantel administered to the mammal over a course of about a 30 day period does not exceed 25 mg of monepantel per kg of mammal body weight.

Embodiment 95: The medicated feed for use in the method according to embodiments 93-94, wherein the amount of monepantel in the micro-dose is in a range selected from the group consisting of: about 0.025 mg/kg to about 0.83 mg/kg; about 0.05 mg/kg to about 0.5 mg/kg; about 0.125 mg/kg to about 0.25 mg/kg.

Embodiment 96: The medicated feed for use in the method according to embodiment 93-95, wherein the amount of montepantel in the micro-dose is selected from the group consisting of: about 0.025 mg/kg; about 0.05 mg/kg; about 0.125 mg/kg; about 0.25 mg/kg; about 0.5 mg/kg; and about 0.83 mg/kg.

Embodiment 97: The medicated feed for use in the method according to embodiments 77-96, wherein the number of days between administering any two micro-doses is less than a period of 2 days, 3 days, 4 days, 5 days, 6 days or 7 days.

Embodiment 98: The medicated feed for use in the method according to embodiments 77-97, wherein the at least one micro-dose is administered orally.

Embodiment 99: The medicated feed for use in the method according to embodiments 77-98, further comprising administering at least one additional anthelmintic.

Embodiment 100: The medicated feed for use in the method according to embodiment 99, wherein the at least one additional anthelmintic is a systemic anthelmintic.

Embodiment 101: The medicated feed for use in the method according to embodiments 99-100, wherein the at least one additional anthelmintic has a half-life of less than 1 day.

Embodiment 102: The medicated feed for use in the method according to embodiment 99, wherein the at least one additional anthelmintic is selected from the group consisting of: at least one compound that blocks acetylcholine receptors; at least one compound that acts on the nicotinic acetylcholine receptor ACR-23, and at least one compound that acts on the calcium-gated potassium channel SLO-1.

Embodiment 103: The medicated feed for use in the method according to embodiment 99, wherein the at least one additional anthelmintic is at least one compound selected from the group consisting of: a macrocylic lactone or a salt or a derivative thereof; a cyclooctadepsipeptide or a salt or a derivative thereof; a tetrahydropyrimidine or a salt or a derivative thereof; an imidazothiazole or a salt or a derivative thereof; a paraherquamide or a salt or a derivative thereof; an aminophenylamidine or a salt or a derivative thereof; an organophosphates or a salt or a derivative thereof; a substituted phenyl or a salt or a derivative thereof; a piperazinone or a salt or a derivative thereof; and an octadepsipeptide or a salt or a derivative thereof.

Embodiment 104: The medicated feed for use in the method according to embodiment 103, wherein the at least one additional anthelmintic includes macrocyclic lactone and the macrocyclic lactone is at least one compound selected from the group consisting of: abamectin, doramectin, emamectin, eprinomectin, ivermectin, milbemycin oxime, moxidectin, nemadectin, selamectin; the benzimidazoles and probenzimidazoles, such as albendazole, albendazole-sulphoxide, cambendazole, cyclobendazole, febantel, fenbendazole, flubendazole, mebendazole, netobimin, oxfendazole, oxibendazole, parbendazole, thiabendazole, thiophanate, and triclabendazole and a salt or a derivative of any of the foregoing.

Embodiment 105: The medicated feed for use in the method according to embodiment 103, wherein the at least one additional anthelmintic includes cyclooctadepsipeptide and the cyclooctadepsipeptide is at least one compound selected from the group consisting of: emodepside, PF1022 and a salt or a derivative of any of the foregoing.

Embodiment 106: The medicated feed for use in the method according to embodiment 103, wherein the at least one additional anthelmintic includes amino-acetonitrile derivative and the amino-acetonitrile derivative is monepantel or a salt or a derivative thereof.

Embodiment 107: The medicated feed for use in the method according to embodiment 103, wherein the at least one additional anthelmintic includes tetrahydropyrimidine and the tetrahydropyrimidine is at least one compound selected from the group consisting of: morantel, pyrantel, and oxantel and a salt or a derivative of any of the foregoing.

Embodiment 108: The medicated feed for use in the method according to embodiment 103, wherein the at least one additional anthelmintic includes imidazothiazole and the imidazothiazole is at least one compound selected from the group consisting of: butamisole, levamisole, tetramisole and a salt or a derivative of any of the foregoing.

Embodiment 109: The medicated feed for use in the method according to embodiment 103, wherein the at least one additional anthelmintic includes paraherquamide and the paraherquamide is at least one compound selected from the group consisting of: derquantel, paraherquamide and a salt or a derivative of any of the foregoing.

Embodiment 110: The medicated feed for use in the method according to embodiment 103, wherein the at least one additional anthelmintic includes aminophenylamidine and the aminophenylamidine is at least one compound selected from the group consisting of: amidantel, deacylated amidantel (dAMD), tribendimidine and a salt or a derivative of any of the foregoing.

Embodiment 111: The medicated feed for use in the method according to embodiment 103, wherein the at least one additional anthelmintic includes organophosphate and the organophosphate is at least one compound selected from the group consisting of: coumaphos, crufomate, dichlorvos, haloxone, naphthalofos, trichlorfon and a salt or a derivative of any of the foregoing.

Embodiment 112: The medicated feed for use in the method according to embodiment 103, wherein the at least one additional anthelmintic includes substituted phenyl and the substituted phenyl is at least one compound selected from the group consisting of: bithionol, disophenol, hexachlorophene, niclofolan, meniclopholan, nitroxynil and a salt or a derivative of any of the foregoing.

Embodiment 113: The medicated feed for use in the method according to embodiment 103, wherein the at least one additional anthelmintic includes piperazinone and the piperazinone is at least one compound selected from the group consisting of: praziquantel, epsiprantel and a salt or a derivative of any of the foregoing.

Embodiment 114: The medicated feed for use in the method according to embodiment 99, wherein the at least one additional anthelmintic is a compound selected from the group consisting of: amoscanate, bephenium, bunamidine, clonazepam, clorsulon, diamfenetid, dichlorophen, diethylcarbamazine, emetine, hetolin, hycanthone, lucanthone, Miracil, mirasan, niclosamide, niridazole, nitroxynil, nitroscanate, oltipraz, omphalotin, oxamniquin, paromomycin, piperazine, resorantel, salicylanilides, bromoxanide, brotianide, clioxanide, closantel, niclosamide, oxyclozanide, rafoxanide, tribromsalan and a salt or a derivative of any of the foregoing.

Embodiment 115: The medicated feed for use in the method according to embodiment 99, wherein the at least one additional anthelmintic is diethylcarbamazine or a salt or a derivative thereof.

### BRIEF DESCRIPTION OF THE FIGURES

FIG. 1 is a sample graph of the levels of moxidectin in the plasma of mammals over time (days) given different administration regimens: 1 large dose every 30 days (squares), a daily dose of 0.0008 mg/kg/day (solid line), or daily dose of 0.0016 mg/kg/day (triangles).

### DESCRIPTION

Surprisingly, it has been discovered by the inventors that oral treatments with anthelmintics can provide improved control over parasitic worm infections in mammals when orally administered in smaller, more frequent/chronic doses. The administration is discussed below as being combined with feed. However, it is also contemplated that an anthelmintic may be administered by itself or in a dosage form other than feed, such as a chew, tablet, liquid, gel or other suitable form for oral administration. Advantageously, by using smaller, more frequent doses, less total anthelmintic is required over the same time period to control heartworm infections. Assume, for example, that 24 μg/kg of moxidectin/kg of mammal body weight is needed for a single oral dose in a 30-day (1-month) period according to the prior art approach to reach and maintain a therapeutically effective concentration of moxidectin in the canine's blood for continued heartworm prevention and control. With the inventive approach of smaller and more frequent doses, as little as 0.2-0.72 μg of moxidectin/kg of canine body weight may be needed per day, or 6-21.6 μg of moxidectin/kg of canine body weight cumulative over the same 30-day period.

Advantageously, the total amount of anthelmintic required for a therapeutically effective once-monthly dose can be reduced by 2%-87.5% by converting to daily low dose administrations. However, from a practical perspective, at least two problems arise: (1) creating a homogenous feed; and (2) analytical control testing for a very small dose of anthelmintic may be difficult to accomplish. The analytical matrix from feeds can be quite complex and difficult to assay. Assays will be in the parts per million to billion range for some needed dose and feed concentrations. Thus, it is possible that one of skill in the art may opt to increase the daily dose such that the total of the daily doses over the course of one month equals the prior art once-monthly dose or is even higher, for example, 200% of the prior art once-monthly dose. This may be done to help ensure homogeneity as well as increase assay accuracy and decrease analytical variability when administering the dose as part of a feed. In the case of heartworms, the increased dose may also be effective against resistant isolates of heartworm.

The method and composition taught herein have the further advantage of encouraging compliance because the smaller doses of an anthelmintic can be incorporated into a feed. Since owners naturally follow a daily feeding regimen in any event, this makes it less likely that owners will forget or neglect to administer the anthelmintic. Thus, this disclosure provides a method for prolonged control of heartworm in a safer, more compliant, and more effective manner than that achieved with previously known treatment methodologies. All the owner need remember is to feed their pet daily as they normally would. Further, this method may be used to prevent establishment of resistant isolates of heartworm.

Several classes of anthelmintically active compounds are known, including: the macrocyclic lactones, such as abamectin, doramectin, emamectin, eprinomectin, ivermectin, milbemycin oxime, moxidectin, nemadectin, selamectin; the benzimidazoles and probenzimidazoles, such as albendazole, albendazole-sulphoxide, cambendazole, cyclobendazole, febantel, fenbendazole, flubendazole, mebendazole, netobimin, oxfendazole, oxibendazole, parbendazole, thiabendazole, thiophanate, triclabendazole; the cyclooctadepsipeptides, such as emodepside, PF1022; the amino-acetonitrile derivatives, such as monepantel; the tetrahydropyrimidines, such as morantel, pyrantel, oxantel; the imidazothiazoles, such as butamisole, levamisole, tetramisole; the salicylanilides, such as bromoxanide, brotianide, clioxanide, closantel, niclosamide, oxyclozanide, rafoxanide, tribromsalan; the paraherquamides, such as derquantel, paraherquamide; the aminophenylamidines, such as amidantel, deacylated amidantel (dAMD), tribendimidine; the organophosphates, such as coumaphos, crufomate, dichlorvos, haloxone, naphthalofos, trichlorfon; the substituted phenyls, such as bithionol, disophenol, hexachlorophene, niclofolan, meniclopholan, nitroxynil; the piperazinones, such as praziquantel, epsiprantel; and other classes, which include such examples as amoscanate, bephenium, bunamidine, clonazepam, clorsulon, diamfenetid, dichlorophen, diethylcarbamazine, emetine, hetolin, hycanthone, lucanthone, Miracil, mirasan, niclosamide, niridazole, nitroxynil, nitroscanate, oltipraz, omphalotin, oxamniquin, paromomycin, piperazine and resorantel.

Anthelmintics of particular interest for preventing and controlling heartworm infections in mammals are the macrocyclic lactones (avermectins and milbemycins).

The avermectins are a group of 16-membered macrocyclic lactone derivatives. These naturally occurring compounds are fermentation products by Streptomyces avermitilis, a soil actinomycete. One example of an avermectin is ivermectin, shown below:

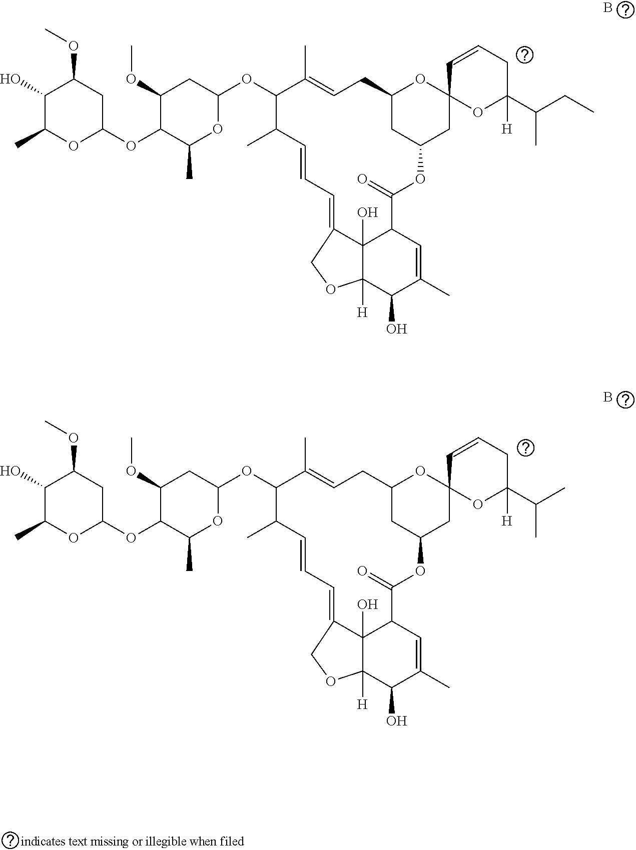

The milbemycins are a group of macrolides first isolated in 1972 from Streptomyces hygroscopicus. They are products of fermentation by Streptomyces species. They have a mechanism of action similar to avermectins and milbemycins often have a longer half-life. For example, moxidectin has a terminal half-life of approximately 13.9 days as compared to a terminal half-life of 1.8 days for ivermectin. Moxidectin, one example of a milbemycin, is shown below:

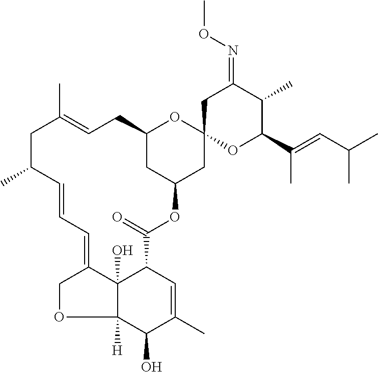 [SMILES: CO/N=C1\C[C@]2(C[C@@H]3C[C@@H](C/C=C(\C)C[C@@H](C)/C=C/C=C4\CO[C@@H]5[C@H](O)C(C)=C[C@@H](C(=O)O3)[C@]45O)O2)O[C@H](/C(C)=C/C(C)C)[C@H]1C]

The amino-acetonitrile derivatives, such as monepantel (N-[(2S)-2-cyano-1-[5-cyano-2-(trifluoromethyl)phenoxy]propan-2-yl]-4-(trifluoromethylsulfanyl)benzamide), are thought to act by interfering with parasite muscle function. Monepantel, its salts, derivatives, metabolites and any biologically active enantiomers thereof are amino-acetonitriles that may be suitable for use with this disclosure.

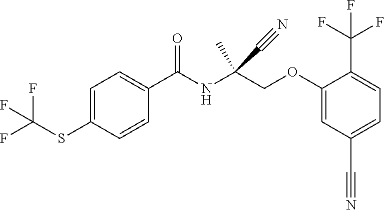 [SMILES: C[C@](C#N)(COc1cc(C#N)ccc1C(F)(F)F)NC(=O)c1ccc(SC(F)(F)F)cc1]

More particularly, anthelmintics with a half-life of at least 1 day are suitable for the methods and formulations of this disclosure. More preferably, anthelmintics with a half-life of at least 3 days are suitable for the methods and formulations of this disclosure. Even more preferably, anthelmintics with a half-life of at least 7 days are suitable for the methods and formulations of this disclosure.

The formulations, or feeds, and methods of this disclosure may further include, in combination with the anthelmintic, one or more other active drug substances having therapeutic efficacy against other types of parasites. Such active substances include agents efficacious against vectors of heartworm transmission, fleas, ticks and intestinal nematode species. Active substances may include, for example, isoxazolines, foramidines, spinosyns, insect growth regulators (including chitin synthesis inhibitors, juvenile hormone analogs, and juvenile hormones) benzimidizoles, and tetrahydropyrimidines.

The methods of this disclosure are carried out by administering the anthelmintic to the mammal in small, frequent doses. To facilitate routine dosing, the anthelmintic administration may be carried out using a feed, chew, tablet, liquid, gel or other suitable form for oral administration. A number of different feeds are envisioned, provided the manufacturing process(es) and feed compositions do not have deleterious effects related to chemical stability, efficacy and safety on the anthelmintic and, if applicable, other active substances. For example, feeds and snacks, chews, treats or supplemental feeds in the broad categories of dry, semi-moist, canned-retorted feeds or fresh refrigerated feeds may be adapted for use with this disclosure. The mammal receives a maintenance quantity of anthelmintic by consuming the feed product on a weekly, semi-weekly or daily basis.

By incorporating smaller doses of anthelmintic into an animal feed composition and administering it at an effective rate (most preferably daily), the blood and tissue levels of the anthelmintic rises over time until it reaches an optimal steady state where it can be maintained by a daily or substantially daily dosage. By contrast, when an anthelmintic is orally administered in larger doses at lower frequency, e.g., a single treatment of a large dose that is administered via “treat or tablet” once in a 30-day period, the level of the anthelmintic in the blood and tissues spikes at the time of the first dose and then declines until the next dose is administered. The administration of a large dose at low frequency means that the mammal must consume more anthelmintic in each dose so that the blood and tissue levels of the anthelmintic does not fall below the necessary level for effective protection before the next dose.

All ratios, percentages, and parts discussed herein are “by weight” unless otherwise specified.

The terms “controlling a parasitic worm infection” and “controlling a helminth infection” refer to preventing, treating, minimizing or eliminating an infection by parasitic worms or helminths in a mammal.

The term “controlling a heartworm infection” refers to preventing, treating, minimizing or eliminating an infection by heartworms in a mammal.

The terms “parasitic worms” and “helminths” are used interchangeably to refer to members of the phyla Annelida, Platyhelminthes, Nematoda and Acanthocephala. The terms “parasitic worms” and “helminths” include the egg, larval and adult stages of development.

The term “heartworm” is used to refer to members of the genus Dirofilaria. The term includes the larval and adult stages of development.

The term “canine” refers to any member of the genus Canis, which includes such species as wolves, dogs, coyotes and jackals.

The term “feline” refers to any member of the subfamily Felidae, which includes such species as the domestic cat, bobcats, wildcats, ocelots, members of the genus lynx, Pallas's cat and cougars.

The term “mustelid” refers to any member of the family Mustelidae, which includes such species as ferrets, American minks, European minks, weasels, badgers and wolverines.

In carrying out the methods of this disclosure, a “feed” is an animal feed, snack, treat or other supplemental feed that may be administered daily or substantially daily. By using different forms of feed, e.g., kibble and treats, a pet owner may vary the mammal's meals and snacks from time to time while still conveniently administering a daily dose of anthelmintic.

The term “chew” refers to a treat that usually has flavor and aromatic properties that are appealing to a mammal, but typically has no nutritional value. In carrying out the methods of this disclosure, a “feed” and/or a “chew” may be used interchangeably.

The term “effective time,” also referred to herein as “effective duration,” for the purposes of this disclosure includes at least the duration of feed administration needed to bring the level of anthelmintic in the mammal's blood or tissues to a sufficiently high level for controlling heartworms, i.e., a “therapeutically effective” level. In some embodiments, the effective time may be as little as three days. In other instances, the effective time may be seven days or fifteen days or longer. As discussed below, the effective time will vary based on how frequently the feed or anthelmintic is administered.

As just alluded, the “effective time” will vary as a function of the frequency at which the feed is administered. The term “effective frequency” as used herein means the number of feedings over a given time that produce a therapeutically effective concentration of anthelmintic in the mammal's blood. In all events, the term “effective frequency” as used herein contemplates multiple feedings including the anthelmintic per month. One of skill in the art will appreciate that the anthelmintic may be administered in a range of frequencies. For example, the anthelmintic may be administered at a frequency of daily, every other day, every third day, once per week or even at inconsistent time intervals.

Further, as discussed above, the effective frequency may affect the duration required to obtain a therapeutically effective level of anthelmintic in the mammal's blood. By way of example, if the mammal were being fed an anthelmintic composition daily, the duration of feed administration required to achieve a therapeutically effective level of anthelmintic in the mammal's blood, and thus the “effective time,” would be comparatively less than if the mammal were being fed the anthelmintic composition only once or twice per week.

Further, the effective frequency is influenced by the amount of the daily dose in mg/kg of body weight of the mammal. Particularly, at slightly higher daily doses, missed doses have less of an impact on efficacy.

Further, the effective frequency is influenced by the duration of treatment. In the initial stages, e.g., before the amount of anthelmintic in the mammal's blood or tissues have reached a therapeutically effective level, the animal feed may need to be administered more often than would be necessary after a longer period of use, i.e., once a therapeutically effective level is obtained.

For purposes of this disclosure, “substantially daily” means a sufficiently regular basis such that the anthelmintic concentration in the mammal's blood or tissues rises to and remains at a therapeutically effective level. For example, the disclosed daily feed composition can preferably be fed to a mammal every day indefinitely. However, as a practical matter, there are many reasons why days may be missed or skipped periodically. For example, the mammal may be ill or the owner may run out of the daily medicated feed composition. The disclosed method is robust enough that the mammal will still be protected from parasitic worms to some extent even with occasional interruptions in daily feeding of the medicated animal feed composition. In carrying out the method of this disclosure, the term “substantially daily” includes at least 10 days per month, more preferably at least 15 days per month, still more preferably at least 20 days per month. All of these feeding frequencies, whether they be, e.g., three times per week, every other day or daily, fit under the umbrella of substantially daily provided that they promote the anthelmintic reaching and maintaining a therapeutically effective level of the anthelmintic in the mammal's blood.

The term “sub-therapeutic” means an amount of an anthelmintic which is, in and of itself, ineffective or sub-optimal for controlling parasitic worm infection in a mammal in a single dose.

The term “therapeutically effective” means that the dose or blood level of an anthelmintic or a physiologically acceptable derivative thereof, or a metabolite thereof, is sufficient to control the helminth infection better than if no drug were present. The anthelmintic or a physiologically acceptable derivative thereof, or a metabolite thereof, may be present on its own or with one or more additional active substances. Preferably it controls the helminth infection at around at least 50% better than if no drug were present, and more preferably it controls the helminth infection at about at least 90% better than if no drug were present.

In carrying out the methods of this disclosure, an effective or therapeutically effective amount of an anthelmintic is administered orally to the mammal. The term “effective amount” or “therapeutically effective amount” refers to the amount needed to control the helminth infection. As those skilled in the art will understand, this amount will vary depending upon a number of factors. These factors include, for example, the type of mammal being treated and its weight and general physical condition, as well as the potency of the anthelmintic.

Anthelmintics vary in potency. Thus, the effective amount of anthelmintic may be calculated for each particular anthelmintic used in at least some of the methods according to this disclosure. In general, the effective amount for a daily dose of an anthelmintic will be in the range of about 25%-90% of the approved label dose for said anthelmintic divided by length of the dosing/retreatment interval (e.g., the dosage divided by 30 for a product administered once per month). One of skill in the art will recognize that a higher dose of, e.g., 90%-200% of the approved label dose for said anthelmintic may be selected for reasons such as, but not limited to, manufacturability, ease of testing and analysis, etc. In some embodiments, the particular dose selected may be sufficient to raise the concentration of said anthelmintic in the mammal's blood to a therapeutically effective level within about 7 days of substantially daily administrations, more preferably within about 5 days of substantially daily administrations, most preferably within about 3 days of substantially daily administrations.

While this disclosure describes concentrations of anthelmintic in terms of feeds such as kibble, it also contemplates administration using other dosage forms, such as treats or chews. It is also contemplated that the anthelmintic may be administered by itself or in a tablet, liquid, gel or other suitable form for oral administration. One of skill in the art will appreciate that the concentration of anthelmintic will vary according to the particular dosage form. For example, where the animal feed is a treat, the concentration of anthelmintic in the treat will be greater than, e.g., the concentration of anthelmintic in a kibble. For example, if the daily dose of anthelmintic based on the weight of the mammal is 10 mg, then a typical 5 g treat may contain about 0.002 percent anthelmintic (by weight). Since the amount of kibble consumed in a day is more than 5 g, the percent anthelmintic in kibble will be smaller.

For example, an effective amount of moxidectin may be a daily or a substantially daily dose of from about 0.2 to about 1.6 μg of moxidectin/kg of body weight of the mammal. Alternatively, an effective amount of moxidectin may be a daily or a substantially daily dose of from about 0.4 to about 1.6 μg of moxidectin/kg of body weight of the mammal. Alternatively, the effective amount of moxidectin may be a daily or a substantially daily dose of from about 0.8 to about 1.6 μg of moxidectin/kg of body weight of the mammal. Alternatively, the effective amount of moxidectin may be a daily or a substantially daily dose of from about 0.2 to about 0.8 μg of moxidectin/kg of body weight of the mammal.

Animal feeds will typically contain from about 0.0000001 to about 0.00008 percent of moxidectin (by weight) in the feed. Preferably between about 0.0000002 to about 0.00005 percent of moxidectin (by weight) in the feed. Most preferably between about 0.000001 to about 0.0001 percent of moxidectin component or components (by weight) in the feed.

In another example, an effective amount of monepantel may be a daily or a substantially daily dose of from about 0.025 to about 0.83 mg of monepantel/kg of body weight of the mammal. Alternatively, an effective amount of monepantel may be a daily or a substantially daily dose of from about 0.05 to about 0.5 mg of monepantel/kg of body weight of the mammal. Alternatively, the effective amount of monepantel may be a daily or a substantially daily dose of from about 0.125 to about 0.5 mg/kg of body weight of the mammal. Alternatively, the effective amount of monepantel may be a daily or a substantially daily dose of from about 0.125 to about 0.25 mg of monepantel/kg of body weight of the mammal.

Animal feeds will typically contain from 0.00005 to about 0.0001 percent of monepantel (by weight) in the feed, more preferably between about 0.0001 to about 0.005 percent of monepantel (by weight) in the feed, most preferably between about 0.0005 to about 0.001 percent of monepantel component or components (by weight) in the feed.

In one aspect, this disclosure relates to a method of controlling a heartworm infection in a mammal by administering a systemically active oral composition including an anthelmintic, or a physiologically acceptable derivative or salt thereof, and animal feed at least once per week, more preferably three times per week, most preferably substantially daily.

In another aspect, this disclosure relates to a systemically active oral composition that includes an anthelmintic and animal feed.

This disclosure also relates to the use of an anthelmintic for the manufacture of an animal feed for controlling a heartworm infection in a mammal.

This disclosure also relates to a method of controlling a heartworm infection in a mammal for a prolonged time, comprising orally administering daily or substantially daily doses of an effective amount of an anthelmintic to the mammal in a daily feed. A daily feed is a feed that is intended to be administered daily, but which may be administered for effective times, as described herein. This method is especially useful for controlling heartworms in a mammal for a prolonged time comprising orally administering substantially daily doses of an effective amount of an anthelmintic to the mammal.

An aspect of this disclosure is the oral administration of an amount of an anthelmintic which is ineffective or sub-optimal for controlling heartworm infection in a mammal if administered at longer intervals, such as once per month. However, with repeated administrations at shorter intervals, e.g., substantially daily, as described herein, efficacious control of heartworm infections is achieved. Ineffective or sub-optimal means that a single dosing, as well as several dosings, results in less than a 50% reduction in the heartworm infection, including no, or substantially no, reduction, as compared to no drug administration at all. This reflects the chronic, rather than acute, administration aspect disclosed herein.

In a first embodiment, a method is disclosed for controlling heartworms in a mammal by orally administering to the mammal a daily feed that includes an effective amount of an anthelmintic for an effective time.

In a second embodiment, an anthelmintic for use in controlling heartworms in a mammal in need thereof is disclosed. The anthelmintic may be present in a daily feed in an effective amount, said daily feed to be administered to said mammal for an effective time to control heartworms.

In a third embodiment, a daily feed for controlling heartworms in a mammal, comprising an effective amount of an anthelmintic when administered for an effective time is disclosed.

In an aspect of any of the embodiments, the mammal may be a canine. More particularly, the canine may be a wolf, coyote or a dog.

In an aspect of any of the embodiments, the mammal may be a feline. More particularly, the feline may be a domestic cat, bobcat, wildcat, ocelot, member of the genus lynx, Pallas's cat or cougar.

In an aspect of any of the embodiments, the mammal may be a mustelid. More particularly, the mustelid may be a ferret, mink, weasel, badger or wolverine.

In an aspect of any of the embodiments, the anthelmintic is a macrocyclic lactone or a salt thereof.

### EXAMPLES

The following examples illustrate the methods of this disclosure:

### Example 1

Efficacy of Moxidectin Administered per os, i.e. by Mouth, to Dogs for the Treatment and Control of Dirofilaria immitis.

Methods: A pool of 12 dogs are to be preliminarily examined to ensure satisfactory health on day-35. On Day-34, a modified Knott's test and/or an adult antigen test may be performed to verify the absence of circulating microfilariae or adult antigens of D. immitis in the blood. Dogs exposed to an anthelmintic or endectocide with activity against D. immitis within 60 days prior to Day-28 are to be excluded.

Dogs are to be weighed between Day-32 and Day-30 and randomly assigned to 1 of 4 treatment groups by weight.

Dogs are to be housed indoors in a mosquito free area to help prevent naturally occurring heartworm infections. Dogs may be pair housed, if compatible, or individually housed until allocation to treatment groups on Day-32 to Day-30. Thereafter, the dogs are to be housed individually. Dogs are to be fed a standard commercially available dog food at the recommended rates and should be allowed access to potable water ad libitum.

Dogs assigned to Groups 1 and 2 are to be inoculated on Day-28 with 50±5 third-stage infective D. immitis larvae. Daily oral dosing with moxidectin is to be started on Day 0 and continued until day 179 for Groups 1 and 2.

Dogs assigned to Groups 3 and 4 are to receive a daily dose of moxidectin beginning on Day-28 and continuing through Day-1. On day 0, after dogs have reached steady state drug levels, the dogs are to be inoculated with 50 +5 third-stage infective D. immitis larvae. Daily dosing with moxidectin is to continue from Day 0 to Day 179.

The daily moxidectin dose for each of the study groups (1-4) should be according to Table 1:

TABLE 1

| | No. of Dogs/ | | Day of | | | |
|---|---|---|---|---|---|---|
| | group (n = | | Inoculation | | | |
| Treatment | 12 for entire | Treatment | with 50 L3 | Anticipated Daily | | |
| Group | study) | Description | of D. immitis | Dose (mg/kg)* | Formulation | Route |
| Group 1 | 3 | Cydectin | −28 | Dose level 1: 0.0008 | API in liquid | Oral |
| | | (moxidectin) | | mg/kg daily starting | formulation | |
| | | | | on Day 0, then daily | | |
| | | | | for Day 1 up to and | | |
| | | | | including Day 179 | | |
| Group 2 | | | −28 | Dose level 2: 0.0016 | | |
| | | | | mg/kg daily starting | | |
| | | | | on Day 0, then daily | | |
| | | | | for Day 1 up to and | | |
| | | | | including Day 179 | | |
| Group 3 | | | 0 | Dose level 1: 0.0008 | | |
| | | | | mg/kg daily for 28 | | |
| | | | | consecutive days | | |
| | | | | (=Days −28 to Day −1), | | |
| | | | | then daily for Day | | |
| | | | | 1 up to and including | | |
| | | | | Day 179 mg/kg | | |
| Group 4 | | | 0 | Dose level 2: 0.0016 | | |
| | | | | mg/kg daily for 28 | | |
| | | | | consecutive days | | |
| | | | | (=Days −28 to Day −1), | | |
| | | | | then daily for Day | | |
| | | | | 1 up to and including | | |
| | | | | Day 179 | | |

*The selected mg/kg dose levels represent ca. 1/30 (Group 1 and 3) and 1/15 (Groups 2 and 4) of the therapeutic monthly administered oral dosage levels for moxidectin (ca. 24 μg/kg) to prevent the establishment of adult heartworm disease and for the treatment of intestinal nematode infections in dogs.

Treatment on each dosing day may be administered by oral gavage to dogs in a fed or fasted state. On each scheduled dosing date from Days-28 (Groups 3 and 4) or Day 0 (Groups 1 and 2) to Day 179, each dog in all Groups are to receive a dose of moxidectin based on each dog's pre-treatment or most recent body weight. Dogs in all Groups may be re- weighed on Days 0, 30, 60, 90, 120, 150 and to adjust their daily oral moxidectin oral liquid volumes based on their most recent body weight.

Heartworms, if found, are to be recovered and counted from all study dogs on or after Day 180.

The instant study demonstrates that doses of moxidectin (0.8μg/kg and 1.6 μg/kg) administered to dogs exposed to heartworm and dogs not exposed to heartworm will be effective in treating and/or preventing heartworm. The expected result is 0 heartworms recovered from all 12 dogs.

Using the same study method as described above, but continuing the daily administration of moxidectin in treatment groups 2 and 3 for 90 days, blood may be drawn approximately daily after the initial dose of moxidectin is administered. The average concentration of moxidectin in the blood for different dosage levels can then be determined.

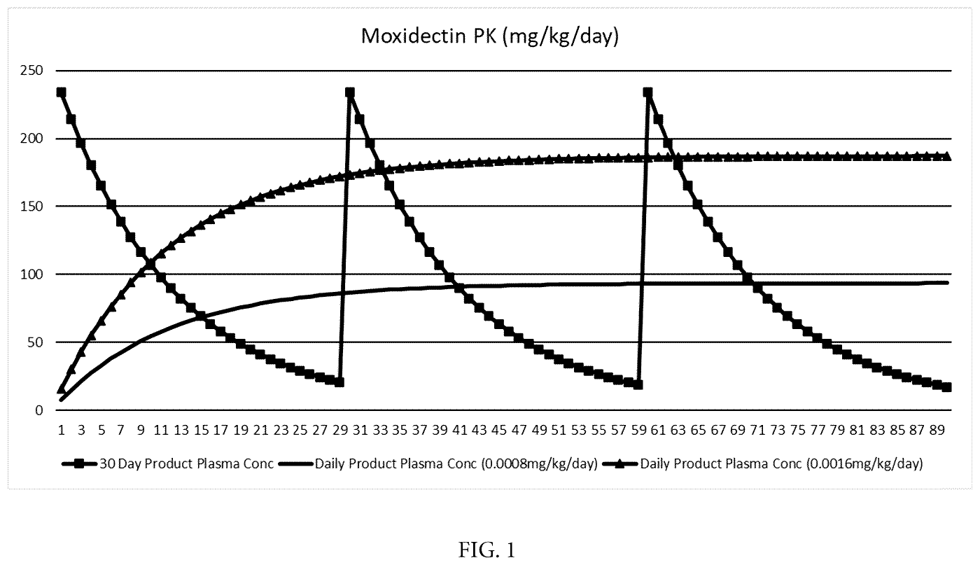

Predicted results of the average plasma concentration of moxidectin in a mammal's blood at the different dosage levels are shown in FIG. 1. The approximate plasma concentration for a daily dose of 0.0008 mg of moxidectin/kg body weight of mammal is shown in a solid line and the approximate plasma concentration for a daily dose of 0.0016 mg of moxidectin/kg body weight of mammal is shown in triangles. For comparison, the approximate plasma concentration for a mammal given a single large dose once every 30 days is also shown (squares) in FIG. 1.

### Example 2

Efficacy of Monepantel Administered per os, i.e. by Mouth, to Dogs for the Treatment and Control of Dirofilaria immitis.

Methods: A pool of 12 dogs are to be preliminarily examined to ensure satisfactory health on day-35. On Day-34, a modified Knott's test and/or an adult antigen test may be performed to verify the absence of circulating microfilariae or adult antigens of D. immitis in the blood. Dogs exposed to an anthelmintic or endectocide with activity against D. immitis within 60 days prior to Day-28 are to be excluded.

Dogs are to be weighed between Day-32 and Day-30 and randomly assigned to 1 of 4 treatment groups by weight.

Dogs are to be housed indoors in a mosquito free area to help prevent naturally occurring heartworm infections. Dogs may be pair housed, if compatible, or individually housed until allocation to treatment groups on Day-32 to Day-30. Thereafter, the dogs are to be housed individually. Dogs are to be fed a standard commercially available dog food at the recommended rates and should be allowed access to potable water ad libitum.

Dogs assigned to Groups 1 and 2 are to be inoculated on Day-28 with 50±5 third-stage infective D. immitis larvae. Daily oral dosing with monepantel is to be started on Day 0 and continued until day 179 for Groups 1 and 2.

Dogs assigned to Groups 3 and 4 are to receive a daily dose of monepantel beginning on Day-28 and continuing through Day-1. On day 0, after dogs have reached steady state drug levels, the dogs are to be inoculated with 50 +5 third-stage infective D. immitis larvae. Daily dosing with monepantel is to continue from Day 0 to Day 179.

The daily monepantel dose for each of the study groups (1-4) should be according to Table 2.

TABLE 2

| | No. of Dogs/ | | Day of | | | |
|---|---|---|---|---|---|---|
| | group (n = | | Inoculation | | | |
| Treatment | 12 for entire | Treatment | with 50 L3 | Anticipated Daily | | |
| Group | study) | Description | of D. immitis | Dose (mg/kg)* | Formulation | Route |
| Group 1 | 3 | Monepantel | −28 | Dose level 1: 0.083 | API in liquid | Oral |
| | | | | mg/kg daily starting | formulation | |
| | | | | on Day 0, then daily | | |
| | | | | for Day 1 up to and | | |
| | | | | including Day 179 | | |
| Group 2 | | | −28 | Dose level 2: 0.83 | | |
| | | | | mg/kg daily starting | | |
| | | | | on Day 0, then daily | | |
| | | | | for Day 1 up to and | | |
| | | | | including Day 179 | | |
| Group 3 | | | 0 | Dose level 1: 0.083 | | |
| | | | | mg/kg daily for 28 | | |
| | | | | consecutive days | | |
| | | | | (=Days −28 to Day −1), | | |
| | | | | then daily for Day | | |
| | | | | 1 up to and including | | |
| | | | | Day 179 mg/kg | | |
| Group 4 | | | 0 | Dose level 2: 0.83 | | |
| | | | | mg/kg daily for 28 | | |
| | | | | consecutive days | | |
| | | | | (=Days −28 to Day −1), | | |
| | | | | then daily for Day | | |
| | | | | 1 up to and including | | |
| | | | | Day 179 | | |

Treatment on each dosing day may be administered by oral gavage to dogs in a fed or fasted state. On each scheduled dosing date from Days-28 (Groups 3 and 4) or Day 0 (Groups 1 and 2) to Day 179, each dog in all Groups are to receive a dose of monepantel based on each dog's pre-treatment or most recent body weight. Dogs in all Groups may be re-weighed on Days 0, 30, 60, 90, 120, 150 and to adjust their daily monepantel oral liquid volumes based on their most recent body weight.

Heartworms, if found, are to be recovered and counted from all study dogs on or after Day 180.

The expected result is 0 heartworms recovered from all 12 dogs, demonstrating that doses of monepantel (0.083 mg/kg and 0.83 mg/kg) administered to dogs exposed to heartworm and dogs not exposed to heartworm will be effective in treating and/or preventing heartworm.

While this invention has been described as having an exemplary design, the present invention may be further modified within the spirit and scope of this disclosure. This application is therefore intended to cover any variations, uses, or adaptations of the invention using its general principles.

What is claimed is:

1. A method of controlling heartworm infections, comprising:
administering a cumulative therapeutically effective amount of a least one systemic anthelmintic to a mammal in need thereof, the cumulative therapeutically effective amount comprising a plurality of individual micro-doses of the at least one systemic anthelmintic,
wherein the micro-doses are administered to the mammal daily or substantially daily, wherein the at least one anthelmintic has a half-life in the mammal of at least at least 4 days, or at least 5 days, or at least 6, days or at least 7, or at least 10 days.

2. The method according to claim 1, wherein the systemic anthelmintic is an integral part of at least one of a medicated feed, a chew, a snack, a tablet, a capsule, a powder, a slave, a suspension, a solution, and a paste.

3. The method according to claim 1, wherein the cumulative therapeutically effective amount is sufficient to control an infection of at least one helminth selected from the group consisting of: Dirofilaria immitis, Aelurostrongylus abstrusus, Angiostrongylus vasorum, Crenosoma vulpis and Eucoleus aerophilu.

4. The method according to claim 1, wherein the systemic anthelmintic is a milbemycin.

5. The method according to claim 4, wherein the milbemycin is moxidectin or a therapeutically acceptable salt thereof.

6. The method according to claim 5, wherein the amount of moxidectin in the micro-dose is in a range selected from the group consisting of: about 0.2 μg/kg to about 1.6 μg/kg; about 0.4 μg/kg to about 1.2 μg/kg; about 0.6 μg/kg to about 1.0 μg/kg, about 0.8 μg/kg to about 0.9 μg/kg.

7. The method according to claim 1, wherein the systemic anthelmintic is an amino-acetonitrile derivative or a therapeutically acceptable salt thereof.

8. The method according to claim 7, wherein the amino-acetonitrile derivative is monepantel or a therapeutically acceptable salt thereof.

9. The method according to claim 8, wherein the amount of monepantel in the micro-dose is in a range selected from the group consisting of: about 0.025 mg/kg to about 0.83 mg/kg; about 0.05 mg/kg to about 0.5 mg/kg; about 0.125 mg/kg to about 0.25 mg/kg.

10. The method according to claim 1, wherein the number of days between administering any two micro-doses is less than a period of 2 days, 3 days, 4 days, 5 days, 6 days or 7 days.

11. The method according to claim 1, further comprising administering at least one additional anthelmintic.

12. The method according to claim 11, wherein the at least one additional anthelmintic has a half-life of less than 1 day.

13. A composition for controlling heartworm infections, comprising:
a micro-dose of at least one systemic anthelmintic, said at least one systemic anthelmintic being active in a mammal and having a half-life in the system of the mammal of at least 4 days, or at least 5 days, or at least 6, days or at least 7, or at least 10 days, and a cumulative dose of a plurality of said micro-doses is sufficient to control a heartworm infection.

14. The composition according to claim 13, wherein the systemic anthelmintic is an integral part of at least one formulation selected from the group consisting of: a medicated feed, a treat, a chew, a snack, a tablet, a capsule, a powder, a salve, a suspension, a solution, a sachet, and a paste, or an integral part thereof.

15. The composition according to claim 13, wherein the systemic anthelmintic is a milbemycin.

16. The composition according to claim 13, wherein the systemic anthelmintic is an amino-acetonitrile derivative or a therapeutically acceptable salt thereof.

17. A medicated feed for use in a method of controlling heartworm infections, comprising:
administering a cumulative therapeutically effective amount of a least one systemic anthelmintic to a mammal in need thereof, the cumulative therapeutically effective amount comprising a plurality of individual micro-doses of the at least one systemic anthelmintic,
wherein the micro-doses are administered to the mammal daily or substantially daily, wherein the at least one anthelmintic has a half-life in the mammal of at least at least 4 days, or at least 5 days, or at least 6, days or at least 7, or at least 10 days.

18. The medicated feed for use in the method according to claim 17, wherein the systemic anthelmintic is a milbemycin.

19. The medicated feed for use in the method according to claim 17, wherein the systemic anthelmintic is an amino-acetonitrile derivative or a therapeutically acceptable salt thereof.

20. The medicated feed for use in the method according to claim 17, further comprising administering at least one additional anthelmintic.
